(19) United States
(12) Patent Application Publication
(10) Pub. No.: US 2024/0342458 A1
(43) Pub. Date: Oct. 17, 2024

(54) Intraluminal Disinfecting Needless Connector

(71) Applicant: Halkey-Roberts Corporation, St. Petersburg, FL (US)

(72) Inventor: Jeffery John Albertsen, McKinney, TX (US)

(73) Assignee: Halkey-Roberts Corporation, St. Petersburg, FL (US)

(21) Appl. No.: 18/078,699

(22) Filed: Dec. 9, 2022

Related U.S. Application Data
(60) Provisional application No. 63/287,637, filed on Dec. 9, 2021.

(51) Int. Cl. A61M 39/16; A61M 39/00; A61M 39/02; A61M 39/06

(52) U.S. Cl. CPC A61M 39/162; A61M 39/02; A61M 2039/0009; A61M 2039/0205; A61M 2039/064; A61M 2205/0205; A61M 2205/0216

(57) ABSTRACT

A needleless connector including a valve stem operatively secured with a valve body by a vale cap, the valve body including a passageway, the valve stem including a lumen, such that being accessed by an access device, fluid flows through the lumen and fluid passageway and a tubular segment composed of copper positioned with the fluid flow.

### CROSS-REFERENCE TO RELATED APPLICATIONS

This application claims the benefit of Ser. No. 63/287,637, filed Dec. 9, 2021, the disclosure of which is hereby incorporated by reference herein.

### BACKGROUND OF THE INVENTION

### Field of the Invention

This invention relates to needleless medical connectors commonly employed in intravenous (IV) lines. More particularly, this invention relates to a medical connector having a valve employing an internal elastomeric valve stem.

### Description of the Background Art

Presently there exist numerous types of needleless connectors (commonly straight, T and Y connectors) for fluidly interconnecting intravenous (IV) and other lines. To preclude fluid flow through the connector when not being accessed, some needleless connectors employ a normally-closed valve. Common valves comprise a valve body with an internal elastomeric valve stem positioned therein. Instructions For Use (IFU) require that the upper surface of the valve stem (exposed from the upper surface of the valve body) be “swabbed” by an alcohol pad to sterilize the same prior to being accessed by an access device, such as the tip of a male Luer fitting on the end of a syringe or other IV component.

Elastomeric valve stems typically comprise a through-slit that is forced normally closed when positioned within the valve body and not being accessed (i.e., at rest). During accessing, the tip of the access device forces the valve stem inwardly into the valve body allowing the tip to force itself into the slit to open the valve stem, thereby allowing fluid flow from the access device, through its tip and then through the now forced-open slit.

Upon removal of the tip of the access device from the valve, the elastomeric properties of the valve stem resiliently move the valve stem outwardly to return to its forced, normally closed, at rest, position within the valve body.

Representative patents and patent applications that teach needleless valves having elastomeric valve stems are disclosed in U.S. Pat. Nos. 6,651,956, 6,089,541, 6,036,171 & 9,839,774 and U.S. Published Patent Applications 2008/0009822 and 2005/0261637, the disclosures of which are hereby incorporated herein.

As can be appreciated with all connectors with swabable valves, inadequate swabbing prior to accessing increases the risk of undesired microbiological growth and hence infection inside the valve. Swabbing of the exposed upper surface of the valve stem with an alcohol pad to sterilize the same prior to being accessed by an access device pursuant to the IFU, may sometimes occur too briefly or not vigorously enough (if at all) such that the exposed upper surface is not adequately sterilized, leading to possible microbiological growth and contamination inside of the valve. The resulting hospital-born infections are detrimental and therefore there exists a need for connectors having antimicrobial properties.

Antimicrobial connectors may include, for example, U.S. Pat. No. 9,878,143 which discloses a luer adapter having a body including an inner surface composed of an antimicrobial material and defining a lumen and first and second ends. A hydrophilic barrier is positioned between the lumen and the antimicrobial material and permits the passage of the antimicrobial material therethrough upon exposure to moisture. See also, U.S. Published Patent Application 2020/0398037 (in some embodiments, the needleless connector may be provided with one or more antimicrobial features such as a chlorhexidine-eluting antimicrobial feature implemented as an insert ring, a coating or a lubricant at one or more locations within a chamber of the valve); Published Patent Application PCT/US94/02327 (connector including an antimicrobial agent molded in synthetic resin); U.S. Pat. No. 10,994,118 (antimicrobial stopcock with an antimicrobial coating or insert provided in a fluid channel whereby fluid flow contacts the antimicrobial coating or an antimicrobial agent is eluted from the antimicrobial coating); U.S. Pat. No. 9,770,580 (blood control IV catheter with antimicrobial properties); Published Patent Application PCT/US2020/046762 (antimicrobial access system); U.S. Published Patent Application 2020/0000972 and Published Patent Application 2016/0287758) (catheter assemblies having an antimicrobial coating such as copper-based layer between a corrosion-preventing layer or having a non-eluting copper coating) and U.S. Pat. No. 5,520,664 (catheter with a long-lasting antimicrobial surface treatment).

Therefore, an object of this invention to provide an improvement which overcomes the aforementioned inadequacies of the prior art devices and provides an improvement which is a significant contribution to the advancement of the antimicrobial needleless connector art.

Another object of this invention is to provide a disinfecting needleless connector.

Another object to this invention is to provide a needleless connector having a normally-closed valve to preclude fluid flow through the connector when not being accessed.

Another object to this invention is to provide a needleless connector having a normally-closed valve with an internal tube segment positioned within the internal fluid flow of the valve, the tube segment having antimicrobial properties such as copper.

Another object of this invention is to provide a needleless connector including a valve having a copper tube segment positioned with the valve stem of the valve of the connector.

Another object of this invention is to provide a needleless connector including a valve having a copper tube segment positioned with the valve body of the connector.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, the invention comprises an intraluminal disinfecting needleless connector comprising a valve body in which is operatively positioned an elastomeric valve stem having a through-slit allowing accessing by an access device such as a syringe. In addition, an antimicrobial tubular segment, such as a segment of antimicrobial copper. In one embodiment, the antimicrobial tubular segment is rigidly centered concentrically within the lumen of the valve stem (hereinafter “valve stem embodiment”). In another embodiment, the antimicrobial tubular segment is rigidly secured within the central fluid passageway of the connector immediately downstream of the valve stem (hereinafter “in-passageway embodiment).

Upon accessing, the tip of the access device, such as a syringe, is pushed inwardly into the exposed end of the valve stem forcing it inward to force open the through-slit, thereby allowing fluid flow to/from the syringe through the antimicrobial tubular segment and out the connector. Importantly, once accessing is complete including after possible flushing with a lock solution, any residual fluid in the valve stem in the case of the in-valve stem embodiment or fluid passageway in the case of the in-passageway embodiment, remains exposed to the antimicrobial tubular segment, thereby decreasing the incidence of site inflections.

In regard to the valve stem embodiment, preferably the outside wall of the valve stem and its interior lumen are concentrically circular cylindrical during manufacturing. Also preferably, the outside diameter of the antimicrobial tubular segment is appreciably less than the inside diameter of the lumen of the valve stem to define a circular cylindrical annular space therebetween during assembly of the tubular segment and the valve stem into the valve body. In this manner, during use, should any fluid be trapped between the outside diameter of the antimicrobial tubular segment and the inside diameter of the lumen of the valve stem, such trapped fluid also remains exposed to the antimicrobial tubular segment, thereby further decreasing the incidence of site inflections.

Without departing from the spirit and scope of this invention, the connector of the invention may comprise a T-connector, Y-connector, straight connector, or any other desired configuration of a connector that employs an elastomeric valve stem.

Preferably the through-slit in the valve stem comprises a generally straight through-slit as shown in U.S. Pat. No. 6,651,956 in which the slit is normally-closed when uncompressed but is forcibly squeezed tighter closed due to the compression of the valve stem when inserted into the valve body. Alternatively, the valve stem may comprise a normally-open slit, such as disclosed in U.S. Pat. No. 5,699,821, the disclosure of which is hereby incorporated by reference herein, which is then forcibly squeezed closed when the valve stem is inserted into the valve body.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a top right perspective view of the valve stem embodiment of the needleless connector of the invention;

FIG. 2 is a top left cross-sectional perspective view of the valve stem embodiment of the needleless connector of the invention;

FIG. 3 is front view of the valve stem embodiment of the needleless connector of the invention;

FIG. 4 is rear view of the valve stem embodiment of the needleless connector of the invention;

FIG. 5 is a top view of the valve stem embodiment of the needleless connector of the invention;

FIG. 6 is a bottom view of the valve stem embodiment of the needleless connector of the invention;

FIG. 7 is a right side view of the needleless connector of the invention, the left side view thereof being a mirror image thereof;

FIG. 8 is a cross-sectional view taken along lines 8-8 of FIG. 5;

FIG. 9 is a cross-sectional view of the elastomeric stem of the invention;

FIG. 10 is a longitudinal cross-sectional view of the valve body of the valve stem embodiment of the invention; and

FIG. 11 is a longitudinal cross-sectional view of the in-passageway embodiment of the connector.

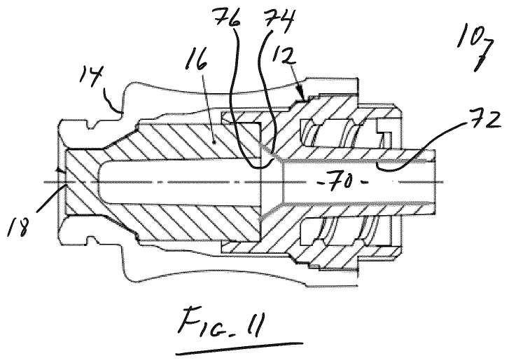

Similar reference characters refer to similar parts throughout the several views of the drawings.

### DETAILED DESCRIPTION OF THE PREFERRED EMBODIMENT

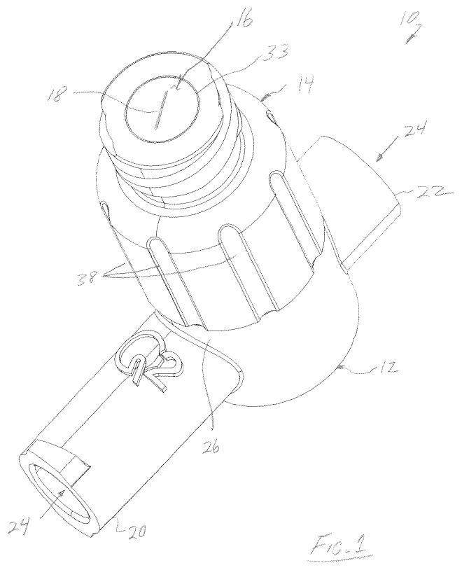

Referring to FIG. 1, the medical connector 10 of the invention comprises a valve body 12, an apertured valve cap 14 and a valve stem 16 having a through-slit 18 allowing accessing by an access device such as a syringe (not shown).

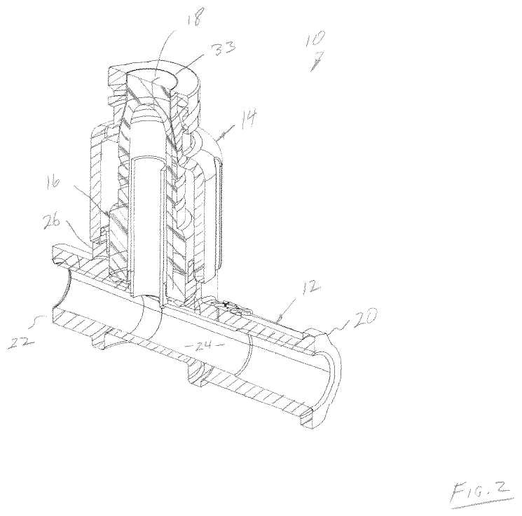

As better shown in FIG. 2, one end 20 or the other end 22 of the valve body 12 may comprise any suitable fitting for connection into an IV set. For example one end 20 may comprise a conventional female luer fitting and the other end 22 may comprise a conventional tube connection fitting. A longitudinal fluid passageway 24 extends from one end 20 to the other 22.

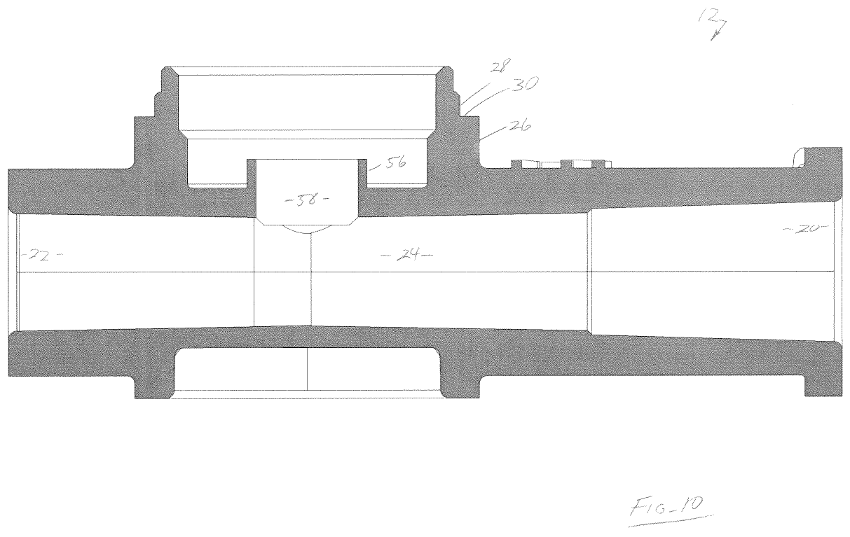

As best shown in FIG. 10, valve body 12 comprises an integrally-molded upstanding boss 26 extending in fluid communication with the sidewall of the longitudinal fluid passageway 24. Boss 26 is illustrated as extending approximately 90° from the fluid passageway 24 forming a T-connector; however, it should be appreciated that the angle may be varied as desired, such as an acute angle as for a Y-connector. Alternatively, the valve body can be provided as a straight, stand-alone valve where the distal end is a male luer connector.

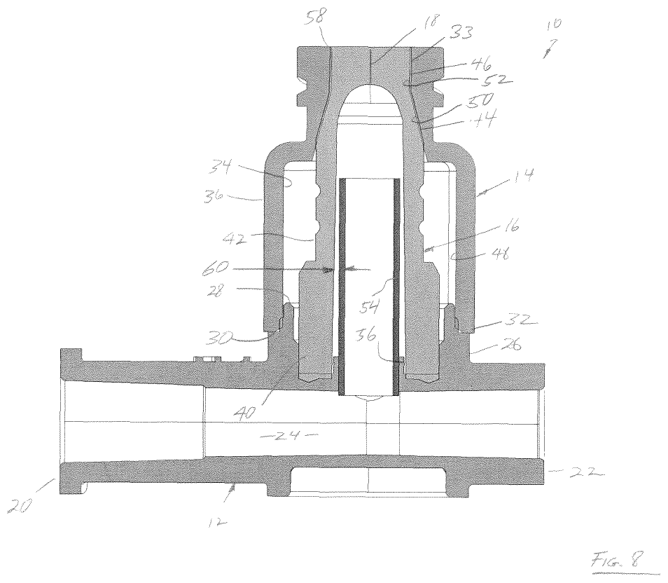

Referring now to FIG. 8, boss 26 comprises a substantially circular cylindrical configuration having a reduced diameter upstanding portion defining a cap seat 30 that is concentrically positioned during assembly within the lowermost circular cylindrical annular edge 32 of the valve cap 14 and allowing permanent assembly therebetween (e.g., sonic or solvent welding or the like).

The apertured cap 14 comprises a central aperture 33 through which the through-slit 18 of uppermost surface of the valve stem 16 is exposed. Preferably, the cap 14 comprises circular cylindrical interior and exterior configurations 34 & 36. As better described below in relation to the valve stem 16, the aperture 33 and interior circular cylindrical configuration 34 is configured and dimensioned to operatively receive the circular cylindrical valve stem 16 therein. The exterior configuration 36 preferably comprises a plurality of longitudinal exterior ribs or indentations 38 to facilitate gripping of the connector 10 during swabbing.

Referring now to FIG. 8, the valve stem 16 comprises a base portion 40 of an increased diameter, preferably configured circular cylindrically, to seal into the boss 26 when assembled therein during manufacturing. Valve stem 16 further comprises a middle portion 42, a frustoconical portion 44 and then a reduced diameter portion 46 preferably extending integrally and circular cylindrically above the base portion 40. Correspondingly, the valve cap 14 preferably comprises a middle portion 48, a frustoconical portion 50 and a reduced diameter portion 52.

The frustoconical portions 44 & 50 are dimensioned and configured to complementary seal against each other when assembled. Indeed, the relative overall length of the portions 40, 42 & 44 relative to portions 48, 50 and 52 may be appreciably less such that the valve stem 16 is under axial compression between the valve cap 14 and valve body 12 when assembled, thereby increasing the sealing between the frustoconical portions 44 & 50. The complementally frustoconical positions 44 & 50 also function to exert some lateral force to more tightly seal the through-slit 18 when not being accessed. The valve stem 16 may comprise one or more annular indentations 62 designed to define the desired bend profile of the middle portion 42.

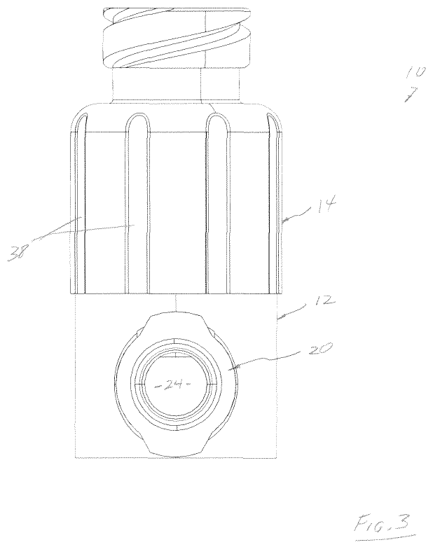

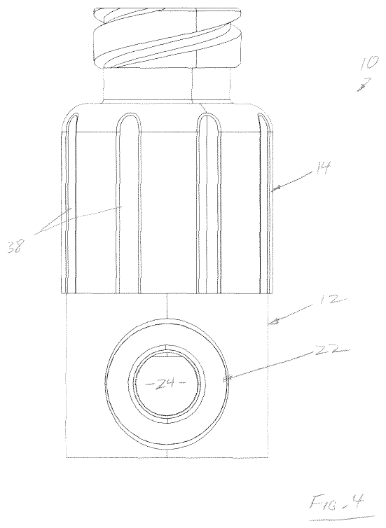

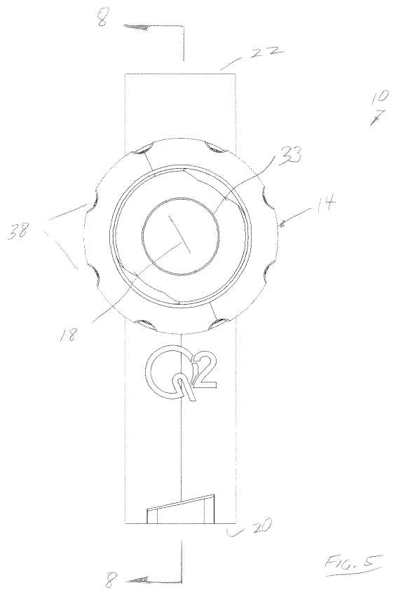

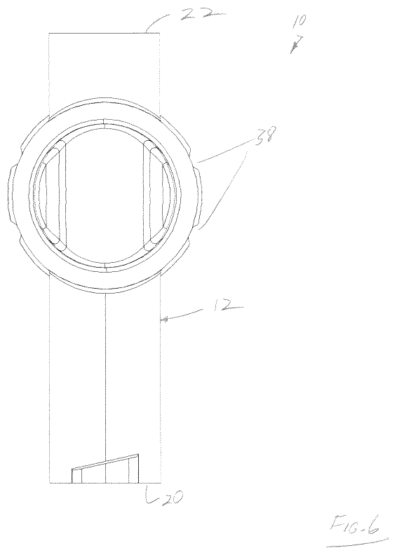

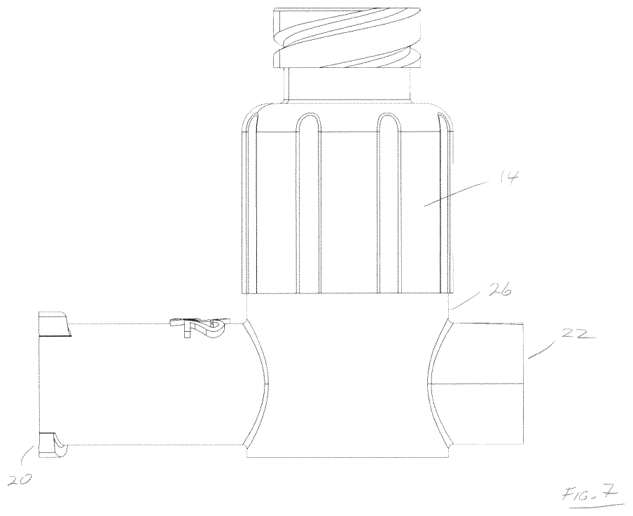

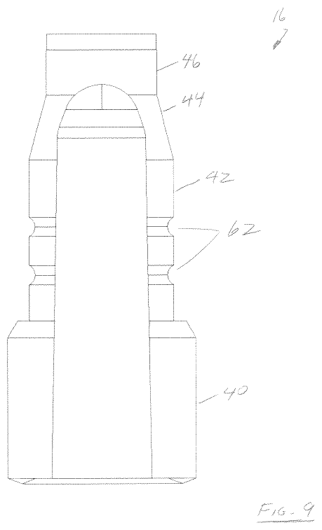

In regard to the valve stem embodiment FIGS. 1-9, the connector 10 further comprises a rigid antimicrobial tubular segment 54, preferably circular cylindrical, composed of an antimicrobial material such as rigid copper or copper alloy. Alternatively, the tubular segment 54 may optionally be composed other suitable antimicrobial materials or impregnated or coated with an antimicrobial agent. Alternatively, the tubular segment may be of a solid, perforated, or coiled configuration.

The tubular segment 54 is fluidly connected through the wall of the longitudinal fluid passageway 24 by press-fitting into a circular cylindrical upstanding mounting flange 56 encircling an access hole 58 in the valve body 12, or other suitable fitment, adhesive or the like, such that during manufacturing assembly, the lumen of the valve stem 16 slipped onto the end of the tubular segment 24 extending from the mounting flange 56. When fully assembled and welded during assembly, the tubular segment 54 is fixedly secured concentrically within the lumen of the valve stem 16.

During accessing by an access device such as a syringe, initial insertion force from the tip of the syringe pushes the reduced diameter portion 46 of the valve stem 16 inwardly. Upon inward movement, the tip of the access device spreads open through slit 18 while simultaneously causing the slit 18 to sealingly encircle (e.g., hug) the uppermost edge of the tubular segment 54 (which may be chamfered or rounded to minimize digging into the lumen of the valve stem 16). Further inward movement, forces the through slit 18 to fully encircle and therefore seal against the outside surface of the tubular 54. Fluid flow to/from the access device is therefore fully exposed to antimicrobial properties of the lumen of the tubular segment 54.

Preferably, the outside diameter of the antimicrobial tubular segment 54 is appreciably less than the inside diameter of the lumen of the valve stem 16 to define a circular cylindrical annular space therebetween (identified by reference numeral 60) during assembly of the tubular segment 54 in the mounting flange 56 of the valve body 12. In this manner, during use, in the event any fluid is trapped between the outside diameter of the antimicrobial tubular segment 54 and the inside diameter of the lumen of the valve stem 16, such trapped fluid remains exposed to the antimicrobial tubular segment 54, thereby further decreasing the incidence of site inflections.

In the valve stem embodiment shown in FIGS. 1-9, the antimicrobial tubular segment 54 comprises a circular cylindrical configuration dimensioned to fit within the lumen of the valve stem 16. In contrast, in the in-passageway embodiment shown in FIG. 10, the antimicrobial tubular segment 70 comprises a generally circular cylindrical portion 72 extending into the longitudinal fluid passageway 24 of the valve body 12 of the connector 10, with the outer diameter of the circular cylindrical portion 72 being approximately equal to the inner diameter of the lumen of the fluid passageway to fit snugly therein.

Preferably, the proximal end of the fluid passageway 24 comprises a chamfered end 74 and the proximal end of the tubular segment correspondingly including a funnel-shaped configuration 76 dimensioned to fit therein. Also preferably, the outer diameter of the rim of the funnel-shaped configuration 76 is greater than the diameter of the lumen of the valve stem 16. Upon assembly during manufacture, the tubular segment 70 is captured within the fluid passageway 24 and is precluded from extending into the lumen of the valve stem 16 (and obviating the need for a mounting flange 56 for securing the tubular segment 54 as in the case of the valve stem embodiment described above).

Importantly, in both embodiments, during use all the fluid flowing through the connector 10 flows through the lumen of the antimicrobial tubular segment 54 or 70, thereby being exposed the antimicrobial effects thereof.

The present invention includes that contained in the appended claims as well as that of the foregoing description. Although this description has been described in its preferred form with a certain degree of particularity, it should be understood that the present disclosure of the preferred form has been made only by way of example and that numerous changes in the details of construction, combination, or arrangement of parts thereof may be resorted to without departing from the spirit and scope of the invention.

Now that the invention has been described,

What is claimed is:

1. A needleless connector comprising in combination:
a valve stem operatively secured with a valve body by a valve cap, the valve body including a fluid passageway, the valve stem including a lumen, such that upon being accessed by an access device, fluid flows through said lumen and said fluid passageway. and
a tubular segment composed of copper positioned within the fluid flow.

2. The needleless connector as set forth in claim 1, wherein said tubular segment is positioned within said lumen of said valve stem.

3. The needleless connector as set forth in claim 2, further including a mounting flange for mounting said tubular segment within said lumen of said valve stem.

4. The needleless connector as set forth in claim 3, wherein an annular space is defined between the outer diameter of said tubular segment and the inner diameter of said lumen of said valve stem.

5. The needleless connector as set forth in claim 2, wherein said valve stem comprises a through-slit which, upon accessing by an access device, said through-slit opens allowing fluid flow through said tubular segment.

6. The needleless connector as set forth in claim 1, wherein said tubular segment is positioned within said fluid passageway of said valve body.

7. The needleless connector as set forth in claim 6, wherein a proximal end of said fluid passageway is chamfered and wherein a proximal end of said tubular segment comprises a funnel-shaped configuration to fit therein.

8. The needleless connector as set forth in claim 6, wherein said valve stem comprises a through-slit which, upon accessing by an access device, said through-slit opens allowing fluid flow through said tubular segment.

9. The needleless connector as set forth in claim 1, wherein said valve stem comprises an elastomeric valve stem.

10. The needleless connector as set forth in claim 1, wherein said valve stem comprises a frustoconical configuration that seals within a corresponding frustoconical configuration of said valve cap.

11. The needleless connector as set forth in claim 1, wherein said valve stem comprises a through-slit exposed from said valve cap capable of being swabbed prior to use.
